(19) United States
(12) Patent Application Publication
(10) Pub. No.: US 2016/0270815 A1
(43) Pub. Date: Sep. 22, 2016

(54) TROCAR ASSEMBLY

(71) Applicant: CARDIFF METROPOLITAN UNIVERSITY, Cardiff (GB)

(72) Inventor: Benjamin Mazur, Bristol Avon (GB)

(21) Appl. No.: 14/442,258

(22) PCT Filed: Nov. 12, 2013
(86) PCT No.: PCT/GB2013/052980

§ 371 Date: May 12, 2015

(30) Foreign Application Priority Data
Nov. 13, 2012 (GB) 1220390.7

(51) Int. Cl. A61B 17/34; A61B 17/02

(52) U.S. Cl. CPC A61B 17/3423; A61B 17/0218; A61B 2017/347; A61B 2017/3456

(57) ABSTRACT

The present invention relates to a trocar assembly comprising a cannula (40) and an obturator (1) arranged, in use, to be received within and extend through the cannula (4). The obturator (1) comprises a shaft (2) and a head (4). The shaft (2) is arranged to extend through the cannula (40) and having an external diameter which is less than the internal diameter of the cannula (40). The head (2) is arranged to protrude beyond the cannula (40). At least a portion of the head (4) has an external diameter which is greater than the internal diameter of the cannula (40). Said portion of the head (4) comprises a radially extending cut-out (18) such that the portion of the head (4) is radially collapsible for withdrawal through the cannula (40). The obturator (1) further comprises a locking member (30) which is received within the cut-out (18) to prevent withdrawal of the obturator (1).

### FIELD OF THE INVENTION

The present invention relates to a trocar assembly and particularly, but not exclusively to a trocar assembly comprising a cannula and an obturator.

### BACKGROUND OF THE INVENTION

Trocar assemblies are medical devices used to provide sterile entry ports into the body. A trocar assembly typically comprises a hollow tube known as a cannula which is inserted into the body, for example into the abdominal cavity. Cannulas are, for example, used for the introduction of surgical instruments and in particular for the introduction of laparoscopic instruments for the use in “keyhole” surgery. A trocar assembly typically further comprises a piercing member used for pushing the cannula into place (typically after an initial small incision has been made). Once the cannula is in the desired position the piercing member is withdrawn through the cannula leaving the cannula ready for use

The piercing member may be referred to as an obturator or trocar, with obturator sometimes used to refer to a blunt tipped piercing device and trocar being used to refer to a sharp tipped piercing device. For clarity, the term obturator is used exclusively herein but is not intended to be limited to blunt ended devices. The skilled person will appreciate that the tip shape may be chosen depending upon the particular application for which the trocar assembly is to be used.

### SUMMARY OF THE INVENTION

According to a first aspect of the present invention, there is provided a trocar assembly comprising a cannula and an obturator arranged, in use, to be received within and extend through the cannula and wherein the obturator comprises:
a shaft arranged to extend through the cannula and having an external diameter which is less than the internal diameter of the cannula;
a head arranged to protrude beyond the cannula, wherein at least a portion of the head has an external diameter which is greater than the internal diameter of the cannula and wherein said portion of the head comprises a radially extending cut-out such that the portion of the head is radially collapsible for withdrawal through the cannula; and
a locking member which is received within the cut-out to prevent withdrawal of the obturator.

The head of the obturator may be moveable between an expanded position and a retracted position. In the expanded position the head comprises at least a portion having a maximum external diameter which is greater than the internal diameter of the cannula. In the retracted position the head comprises a maximum external diameter which is less than the internal diameter of the cannula.

The locking member may physically occupy a portion of the cut-out of the head of the obturator so as to prevent the head from collapsing radially (so as to prevent the head from moving from the expanded position to the retracted position) and therefore to prevent the head from being withdrawn from the cannula. The locking member maintains the head having a maximum external diameter (for example in the expanded position) so as to prevent the obturator from being removed from the cannula. For example the locking member may comprise a portion which is arranged to be received within and engage the cut-out of the head of the obturator causing the head of the obturator to be in the expanded position. When the locking member is not engaged/received within the head of the obturator, the head may on application of pressure, in particular radial pressure, be moveable to the retracted position. For example, on sliding the obturator in a forward direction through the cannula, the pressure applied to the head of the obturator on contact with the cannula may cause the head of the obturator to further collapse inwardly when the head has not received the locking member.

The head may be of any suitable shape or cross-section for the intended use. Preferably, at least a portion of the head has a substantially circular cross-section. Preferably, the portion of the head adjacent, in use, to the cannula (for example the rearward portion of the head) has a substantially circular cross-section. When the head of the obturator is not circular in cross-section, the term “diameter” of the head may refer to the effective/maximum diameter of the cross section.

At least a portion, preferably all of the head of the obturator has a smooth profile, for example a smooth outer surface. Preferably, the head of the obturator in use forms a substantially contiguous outer surface with the cannula, for example the transition between the head of the obturator and the cannula is preferably substantially smooth without any steps, bumps, edges or other obstacles along the length of the obturator and cannula. This has the advantage of reducing the possibility of the trocar assembly slipping during insertion. Slips can be caused by steps or bumps along the trocar assembly (for example at the edge of the obturator and the cannula) as the assembly passes through the tissue. The presence of steps or bumps can cause the force needing the be applied by the surgeon to vary increasing the risk of slips. Slips during insertion can cause fatal injuries.

The head preferably comprises a shoulder such that in the expanded position the shoulder is located towards the rearward end of the head. The shoulder is preferably provided by the portion of the head which has an external diameter which is greater than the internal diameter of the cannula. In use, the shoulder of the head is preferably located proximal to the cannula. The shoulder is preferably dimensioned such that the shaft of the obturator can be received within the cannula and the outer surface of the shoulder of the head forms in use a substantially smooth transition between the head and the outer surface of the cannula. The shoulder may form a substantially radially extending tab. The tab may engage the front end of the cannula.

The head preferably has a forwardly tapering profile extending from a maximum diameter at the rearward end of the head to a minimum diameter at a forward tip. The rearward end of the head is the portion of the head which in use is located proximal to the cannula. The forward end of the head is the portion of the head which in use is placed against the skin of the patient. The head may have any suitable profile depending on the use of the trocar assembly for example the head may have an arrow head profile.

The locking member may be axially moveable relative to the obturator. The shaft of the obturator may provide an opening in the rearward end of the shaft (for example the end furthest away from the head of the obturator). The opening may extend into a channel extending within the shaft. The channel may be arranged to receive the locking member. The channel may extend in a central location within the shaft and extend parallel to the longitudinal axis of the obturator. The locking member may be axially moveable relative to the channel of the obturator. The locking member may be slidable relative to the obturator, for example the locking member may be slidable relative to the shaft or channel of the obturator.

The locking member is preferably moveable between a locked position and an unlocked position. When the locking member is in the locked position at least a portion of the head has an external diameter which is greater than the internal diameter of the cannula. In the locked position, the locking member therefore retains the obturator in an extended position in which a portion of the head has an external diameter which is larger than the internal diameter of the cannula. The locking member therefore maintains the obturator in position relative to the cannula and cannot be withdrawn in a rearward direction through the cannula. When the locking member is arranged to be in the unlocked position, the locking member is disengaged from the obturator and the head may collapse radially such that the maximum external diameter of the head is less than the internal diameter of the cannula. The obturator may therefore be withdrawn through the cannula. The locking member may be removed from at least a portion of the obturator in order to be in the unlocked position. Preferably, the locking member is removed from at least the head of the obturator. For example, to place the locking member in the unlocked position the locking member may be slid in a rearward direction out of contact with the head of the obturator. Removal of the locking member may unlock the obturator, for example allow at least a portion of the head of the obturator to collapse radially to allow removal of the obturator from the cannula. The locking member is preferably moveable between the locked and unlocked positions by sliding the locking member relative to the obturator. For example, to unlock the obturator (for example to disengage the locking member from the obturator) the locking member is preferably slid rearwardly (i.e. in a direction towards the rear of the head and away from the forward tip) relative to the obturator. This arrangement is advantageous as during insertion of the trocar assembly, the surgeon imparts forward pressure onto the assembly. The locking member cannot therefore be accidentally displaced from the locked position to the unlocked position during insertion by the forward pressure applied by the surgeon.

The locking member may comprise an elongate rod arranged, in use, to be received within and extend through the obturator. For example the elongate rod may be arranged in use to be received within and extend through a channel provided by the shaft of the obturator.

The locking member may further comprise at least one radially extending projection at its forward end arranged in use to engage at least a portion of the cut-out of the head of the obturator. The locking member preferably comprises two radially extending spaced apart projections. For example, the locking member may comprise four radially extending spaced apart projections. Preferably, the spaced apart projections are arranged to be radially opposing. The, or each, radially extending projection of the locking member may be arranged to be received in a cut-out in the head of the obturator.

The cut-out preferably comprises a radial slot. The cut-out preferably extends across the full radial width of the obturator. The cut-out preferably comprises a plurality of radial slots arranged to receive the or each radially extending projection of the locking member. Preferably, the cut-out comprises two opposing slots arranged to receive two radially opposing radially extending projections of the locking member. The cut-out may comprise four radially spaced apart slots, for example four substantially orthogonal slots arranged to receive four radially opposing radially extending projections of the locking member. The four spaced apart slots of the cut-out preferably intersect at the central axis of the obturator. The cross section of the obturator may comprises a plurality of segments, for example the obturator may comprise two segments. The two segments may be equal in proportions and therefore the head of the obturator may comprise two halves. The head of the obturator may comprise four segments. The four segments may be equal in proportions and therefore the head of the obturator may comprise four quarters.

The cut-out, for example the radial slot(s) may extend axially along at least a portion of the head. The cut-out, for example the radial slot(s) may extend axially along at least a portion of the shaft of the obturator. The portion of the cut-out which extends along at least a portion of the shaft may herein be referred to as the first slot(s) of the obturator. The cut-out, for example the radial slot(s) are preferably spaced away from the forward tip of the obturator. For example the cut-out, for example the radial slot(s) may be set back from the forward tip of the obturator. The forward tip of the obturator is preferably formed from a solid body. The forward tip of the obturator is preferably unslotted.

The obturator and locking member are preferably provided with cooperating interlocking features such that the locking member may releasably engage the obturator so as to retain the locking member in the locked or unlocked position. The obturator and locking member may comprise cooperating interlocking features so as to provide for twist-lock engagement. Alternatively, the obturator and locking member may comprise cooperating interlocking features so as to provide for snap fit engagement with sprung projections.

The rearward end of the first slot(s) provided by the cut-out located on the shaft may extend into at least one second slot. The at least one second slot may extend at an angle to the first slot(s). Preferably, the at least one second slot extends substantially orthogonal to the direction of the first slot(s) provided by the cut-out. Preferably the at least one second slot extends in a circumferential direction along at least a portion of the shaft. The at least one second slot is arranged to cooperatively engage the locking member. For example, the at least one second slot may be arranged to engage the protrusion(s) of the locking member. Preferably, the at least one second slot engages the locking member to secure the locking member in position and allow the obturator to be in or moved to the retracted position. As such the at least one second slot enables the locking member to be held in position while allowing the head of the obturator to be able to collapse radially to the retracted position on insertion through the cannula. For example, the obturator is inserted through the cannula in the forward direction. The front tip of the head is received within the cannula. As the head is moved forward through the cannula the portion of the head having (in the expanded position) a greater external diameter than the internal diameter of the cannula makes contact with the cannula. Movement of the obturator in the forward direction causes pressure, for example radial pressure, to be exerted on the head of the obturator causing the head to collapse radially allowing the obturator to be inserted through the cannula. The obturator is inserted until the head of the obturator protrudes from the forward end of the cannula (the end of the cannula which is closest in use to the skin of the patient). The surgeon may then twist the locking member relative to the obturator such that the locking member disengages the at least one second slot and engages the first slot(s) provided by the cut-out portion. The locking member may then be slid in a forward direction towards the head of the obturator. The head receives the locking member in the cut-out of the head and as such the head radially expands such that at least a portion of the head has an external diameter which is greater than the internal diameter of the cannula. The head is now placed in the expanded position. Any rearward movement of the obturator causes the portion of the head having a greater external diameter than the internal diameter of the cannula to abut the cannula, thereby preventing unintentional removal of the obturator from the cannula.

According to a second aspect of the invention, there is provided an obturator as described herein for use in a cannula assembly. The obturator is preferably at least partially collapsible.

According to a third aspect of the invention, there is provided an obturator assembly for use in a cannula comprising an obturator and a locking member as described herein.

Whilst the invention has been described above, it extends to any inventive combination of features set out above or in the following description or drawings.

Front as used herein will be understood to refer to the end of the trocar assembly (or components thereof) which faces, in use, towards the body into which the trocar assembly is to be inserted (i.e. the end which is pointed at the skin). Rear as used herein will be understood to refer to the end of the trocar assembly (or components thereof) which, in use, are furthest from the body end of the trocar assembly (i.e. the end which is pointed away from the body and faces the surgeon). Forward and rearward will likewise be understood to refer to the directions orientated towards the front and rear of the trocar assembly.

### BRIEF DESCRIPTION OF THE DRAWINGS

Specific embodiments of the invention will now be described in detail, by way of example only, and with reference to the accompanying drawings in which:

FIG. 1 is a schematic illustration of an obturator according to one embodiment of the invention;

FIG. 2 is a cross-sectional view of the obturator of FIG. 1;

FIG. 3 is a schematic illustration of a locking member according to one embodiment of the invention;

FIG. 4 is a schematic illustration of the obturator of FIG. 1 in a locked position together with the locking member of FIG. 3;

FIG. 5 is a schematic illustration of the trocar assembly according to one embodiment of the invention comprising the obturator of FIG. 1 in a locked position together with the locking member of FIG. 3 and the cannula;

FIG. 6 is a schematic illustration of the forward end of the trocar assembly of FIG. 5.

### DETAILED DESCRIPTION OF EMBODIMENTS

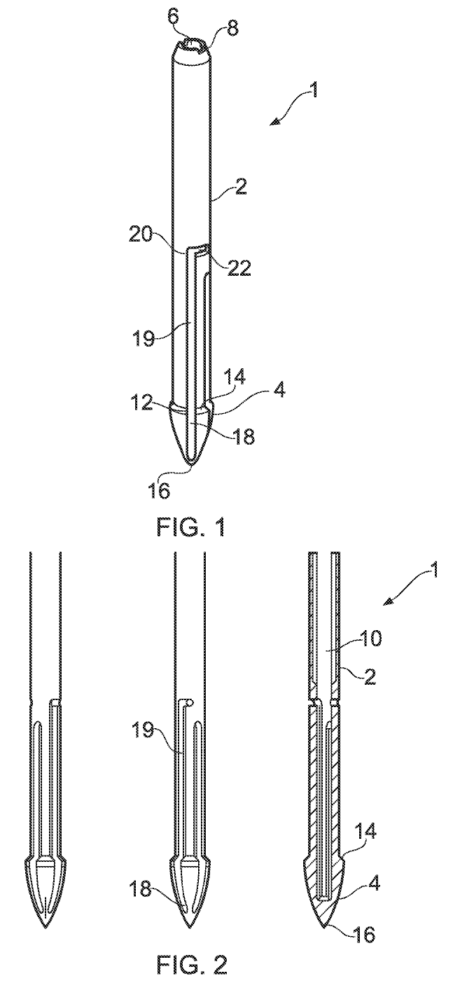

With reference to FIGS. 1 and 2, the trocar assembly comprises a cannula 40 (shown in FIG. 4) and an obturator 1 comprising a shaft 2 connected to a head 4. The shaft 2 is shaped and dimensioned to be received in use within and extend through the cannula 40. The shaft 2 has an external diameter which is less than the internal diameter of the cannula (not shown). The shaft 2 provides a central opening 6 at its rearward end 8. The central opening 6 extends into a channel 10 which extends within the shaft 2 parallel to the longitudinal axis of the shaft 2. The channel 10 extends through the shaft 2 into the head 4.

The head 4 is arranged to protrude beyond the cannula 40. The rearward portion 12 of the head 4 has a substantially circular cross-section. The head 4 is collapsible between an expanded position and a retracted position. In the expanded position, the rearward portion 12 of the head provides a shoulder 14 having a maximum external diameter which is greater than the internal diameter of the cannula 40. In the expanded position, the shoulder 14 of the head 4 has an external diameter which is approximately equal to the external diameter of the cannula 40 so as to provide a smooth profile so as to form a substantially contiguous outer surface with the cannula. In the retracted position, the head 4 has a maximum external diameter which is less than the internal diameter of the cannula (not shown).

The head 4 has a forwardly tapering profile extending from a maximum external diameter at the rearward portion 12 to a minimum external diameter at the forward tip 16. The head 4 is provided with a cut-out 18. The cut-out 18 comprises two radially extending and radially opposed slots. The cut-out 18 extends along a portion of the head 4 and extends rearwardly along the shaft 2 to provide first slots 19. The cut-out 18 is spaced away from the forward tip 16 of the obturator 1. The forward tip 16 is unslotted and is formed from a solid body. The rearward end 20 of the first slots 19 extends into second slots 22. Second slots 22 extend orthogonal to and circumferentially away from the first slots 19. The second slots 22 extend circumferentially about a portion of the shaft 2 (for example 90° around the shaft).

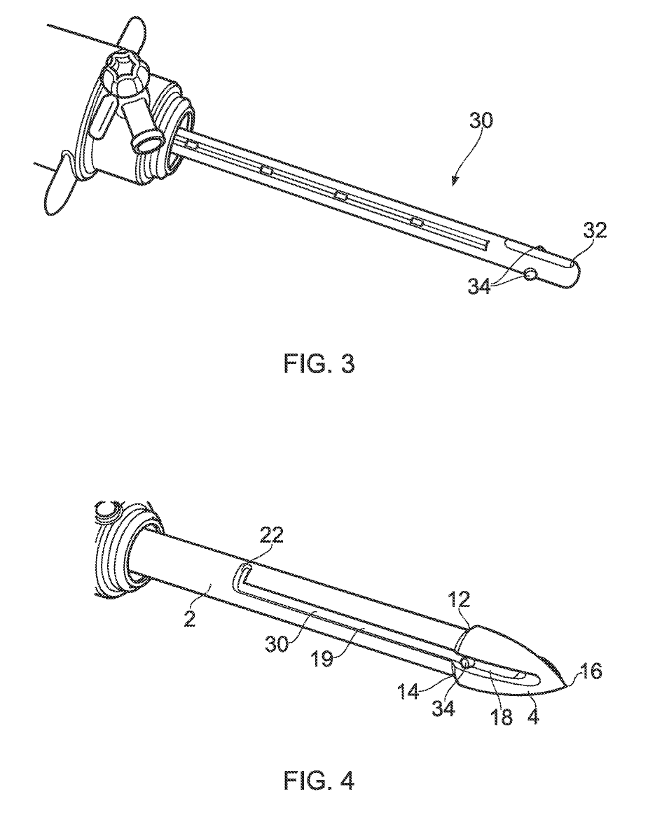

With reference to FIG. 3, the locking member 30 is in the form of an elongate rod. The locking member 30 has a forward end 32 and a rearward end (not shown). The locking member 30 is arranged to be received within the channel 10 of the obturator 1. The locking member 30 comprises two radially extending and radially opposed projections 34 at the forward end 32. The projections 34 are arranged and dimensioned to co-operatively engage in use at least a portion of the first slots 19 of the obturator 1.

With reference to FIGS. 4 to 6 in use, the locking member 30 is inserted into opening 6 and slid forward into the channel 8 provided by the obturator 1. The locking member 30 is arranged initially such that the two radially extending and radially opposed protrusions 34 are engaged with two circumferentially extending slots 22 located on opposing surfaces of the shaft 2. Engagement of the protrusions 34 with slots 22 enables the head 4 of the obturator 1 to be moveable to a retracted position such that the maximum external diameter of the shoulder 14 is less than the internal diameter of the cannula. As a result, the head of the obturator 1 can be inserted into and slid forward through cannula 40. The obturator 1 is inserted through the cannula 40 such that the head 4 protrudes beyond the cannula 40. Locking member 30 is then twisted such that the protrusions 34 disengage slots 22. The locking member 30 is slid in a forward direction through channel 8 of the shaft 2. The protrusions 34 slide forward along the longitudinal portion of slots 18. The protrusions 34 engage the cut-out 18 in the head 4 of the obturator 1. The obturator 1 is now placed in the expanded position such that the head 4 has expanded radially so that the shoulder 14 has a maximum external diameter which is greater than the internal diameter of the cannula. With the locking member 30 in the locked position the head 4 cannot be radially collapsed inwardly since the protrusions 34 are physically located within the cut-out 18 to prevent collapse thereof. As such the head 4 cannot be withdrawn in a rearward direction through the cannula. In the locked position, when the head 4 is in the expanded position, rearward movement of the head 4 causes the shoulder 14 to abut the adjacent end of the cannula. The shoulder 14 is dimensioned such that in the expanded position/locked position the outer surface of shoulder 14 in use forms a substantially smooth transition between the head 4 and the outer surface of the cannula 40.

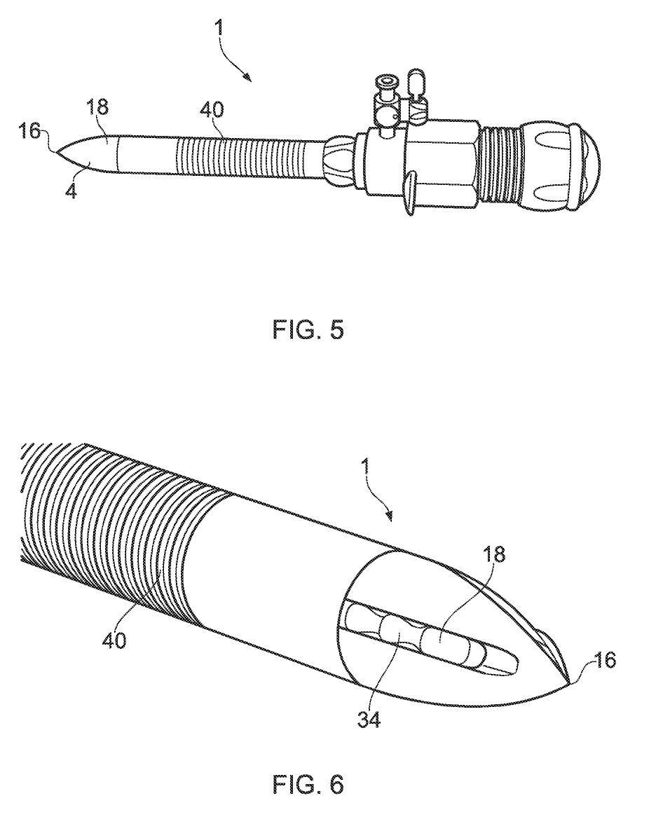

The trocar assembly as shown in FIGS. 5 and 6 is then inserted through the tissue with a forward pressure exerted on the assembly. The locking member 30 cannot be accidentally unlocked or disengaged from the obturator 1 by forward pressure applied by the surgeon since any forward pressure which might be transmitted to the locking member urges it to remain in its forward, locked position. Therefore the assembly has the advantage that the obturator 1 cannot accidentally slip or be removed during insertion through the tissue. Once the cannula is in the desired location, the locking member 30 is removed in a rearward direction from the obturator Removal of the projections 34 from the cut-out 18 enable the head 4 of the obturator 1 to collapse radially inwardly to the retracted position. In the retracted position the head 4 has a maximum external diameter which is less than the internal diameter of the cannula and as such the obturator can be removed through the cannula 40.

1. A trocar assembly comprising a cannula and an obturator arranged, in use, to be received within and extend through the cannula and wherein the obturator comprises:
a shaft arranged to extend through the cannula and having an external diameter which is less than the internal diameter of the cannula;
a head arranged to protrude beyond the cannula, wherein at least a portion of the head has an external diameter which is greater than the internal diameter of the cannula and wherein said portion of the head comprises a radially extending cut-out such that the portion of the head is radially collapsible for withdrawal through the cannula; and
a locking member which is received within the cut-out to prevent withdrawal of the obturator.

2. An assembly as claimed in claim 1, wherein the external diameter of a portion of the head is substantially equal to the external diameter of the cannula.

3. An assembly as claimed in claim 1 or 2, wherein the portion of the head comprises a shoulder at the rearward end of the head.

4. An assembly as claimed in claim 3, wherein the shoulder forms a substantially radially extending tab which may engage the front end of the cannula.

5. An assembly as claimed in any preceding claim, wherein the head has a forwardly tapering profile extending from a maximum diameter at the rear to a minimum diameter at a forward tip.

6. An assembly as claimed in any preceding claim, wherein the locking member is axially moveable relative to the obturator.

7. An assembly as claimed in claim 6, wherein the locking member is slid rearwardly to unlock the obturator.

8. An assembly as claimed in any preceding claim, wherein the obturator and locking member are provided with cooperating interlocking features such that the locking member may be retained in at least one of its locked or unlocked position.

9. An assembly as claimed in any preceding claim wherein the locking member comprises an elongate rod arranged, in use, to be received within and extend through the obturator.

10. An assembly as claimed in claim 9, wherein the locking member further comprises at least one radially extending projection at its forward end arranged to engage at least a portion of the cut-out of the obturator.

11. An assembly as claimed in claim 10, wherein the, or each, radially extending projection is arranged to be received in a slot in the obturator.

12. An assembly as claimed in any preceding claim wherein the cut-out comprises a radial slot.

13. An assembly as claimed in claim 12, wherein the cut-out extends across the full radial width of the obturator.

14. An assembly as claimed in claim 12 or 13, wherein the cut-out comprises a plurality of radial slots.

15. An assembly as claimed in any one of claims 12 to 14, wherein the radial slots extend axially along both the head and shaft of the obturator.

16. An assembly as claimed in any preceding claim wherein the slot is set back from the tip of the obturator.

17. An obturator for use in a cannula assembly as claimed in any preceding claim.

18. An obturator assembly for use in a cannula comprising an obturator and a locking member as claimed in any preceding claim.
